(19) United States
(12) Patent Application Publication
(10) Pub. No.: US 2022/0296787 A1
(43) Pub. Date: Sep. 22, 2022

(54) ANTIMICROBIAL BODY ACCESS SYSTEM

(71) Applicant: PFM MEDICAL INC., Carlsbad, CA (US)

(72) Inventor: Jiaye Jho, Carlsbad, CA (US)

(21) Appl. No.: 17/635,844

(22) PCT Filed: Aug. 18, 2020
(86) PCT No.: PCT/US20/46762

§ 371 Date: Feb. 16, 2022

Related U.S. Application Data
(60) Provisional application No. 62/888,877, filed on Aug. 19, 2019.

(51) Int. Cl. A61L 29/16; A61M 27/00; A61L 29/06

(52) U.S. Cl. CPC A61L 29/16; A61M 27/00; A61L 29/06; A61M 2205/0205; A61L 2300/404; A61L 2300/104; A61L 2300/206; A61L 2300/216

(57) ABSTRACT

An antimicrobial catheter includes an elongated body adapted to be inserted into a body cavity of a patient that is not vascular or the urethra. An antimicrobial surface is positioned along an exterior circumferential surface of the elongated body, wherein the antimicrobial surface is configured to inhibit pathogens. The elongated body has at least one interior lumen defined by a sidewall forming the elongated body. The exterior circumferential surface extends between a first end of the elongated body and a second end of the elongated body.

### CROSS-REFERENCE TO RELATED APPLICATION

This application claims priority to U.S. Patent Application No. 62/888,877, filed Aug. 19, 2019, entitled “VALVE ASSEMBLY FOR BODY ACCESS DEVICE”, the content of which is hereby incorporated by reference herein in its entirety.

### BACKGROUND

A volume of fluid can sometimes gather within a cavity location in a patient's body due to medical conditions such as cancer. In such situations, a medical practitioner can use a catheter or other body cavity access device to drain such fluid from the patient's body. Such a catheter can be a transcutaneously inserted catheter that is introduced into the body cavity such that a first portion, such as a distal end, is positioned in the body cavity of a patient while a second portion, such as a proximal end or proximal region, is positioned transcutaneously outside of a patient's body.

Infection of the tissue at an insertion site or within a body cavity of the patient may occur during the insertion, use, and/or removal of a catheter from the body of a patient. The patient's skin, initially punctured by a needle or other transcutaneous access instrument to allow the insertion of a catheter through the skin and into an existing body cavity (such as the pleural space or abdominal space) may be exposed to infectious agents such as bacteria, viruses, fungi, and other infectious agents disposed on and about the exterior surface of the skin. Such infectious agents may be drawn into the insertion site or body cavity through a contact with the exterior surface of a catheter with the skin, during the insertion, implementation, and removable of the catheter past the skin barrier.

The infectious agents, can lead to inflammation and cell destruction in the tissues surrounding the body cavity, or other remote infection sites.

### SUMMARY

The disclosed device, system and method provides a solution to the shortcomings of such conventional catheters through the provision of an infectious agent-preventing antimicrobial feature that is coupled to the catheter such as via a coating or via a material that is used to manufacture the catheter. The antimicrobial feature thwarts the communication of infectious agents such as bacteria and viruses into the flesh below an insertion site, such as within a pre-existing body cavity in which the catheter is positioned.

In one aspect, there is disclosed an antimicrobial catheter comprising: an elongated body adapted to be inserted into a body cavity of a patient that is not vascular or the urethra, the elongated body having at least one interior lumen defined by a sidewall forming the elongated body, the elongated body having an exterior circumferential surface extending between a first end of the elongated body and a second end of the elongated body; and an antimicrobial surface positioned along the circumferential surface, wherein the antimicrobial surface is configured to inhibit pathogens.

In another aspect, there is disclosed a method of draining fluid from a body cavity, comprising: inserting a catheter into the body cavity, wherein the body cavity is a pre-existing cavity that is present prior to insertion of the catheter, and wherein the catheter comprises: an elongated body adapted to be inserted into a body cavity of a patient, the elongated body having at least one interior lumen defined by a sidewall forming the elongated body, the elongated body having an exterior circumferential surface extending between a first end of the elongated body and a second end of the elongated body; and an antimicrobial surface positioned along the circumferential surface, wherein the antimicrobial surface is configured to inhibit inhibiting pathogens; and using the catheter to drain fluid out of the body cavity.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 shows a schematic representation of a catheter access system configured to allow fluid access to a body cavity from an extracorporeal location.

FIGS. 1A and 2 shows a non-limiting example catheter access system configured to allow fluid access to a body cavity from an extracorporeal location.

FIG. 3 shows the example catheter access system in an exploded state.

FIG. 4 shows a perspective view of a valved connector of the system.

FIG. 5 shows a distal view of the valved connector.

FIG. 6 shows internal components as they are arranged within an outer housing of the valved connector.

FIG. 7 shows the internal components in an exploded state.

FIG. 8 shows the valved connector coupled to a sheath of the access system.

FIG. 9 shows a schematic representation of the collective assembly of the sheath and the valved connector inserted into a patient.

FIGS. 10 and 11 show cross-sectional view of the catheter access system in the region of the valve connector.

FIG. 12 shows a schematic representation of the collective assembly of the sheath, the valved connector, and the catheter inserted into a body cavity of the patient.

### DETAILED DESCRIPTION

Disclosed herein is an antimicrobial coated catheter system for internal, non-vascular body cavities. Such body cavities typically are non-communicating (in a natural state that has not been manually intervened) to the outside of a patient and can be, for example, chest cavities and abdominal cavities although other cavities are within the scope of this disclosure. The cavity can be transcutaneously accessed using a catheter, such as for the purpose of drainage (like pleural or peritoneal drainage) or fluid exchange like peritoneal dialysis. Vascular or urinary antimicrobial catheters already exist but not for these cavity drainage applications.

The catheter can be made of any of a variety of materials and can be coated with an antimicrobial material. In a nonlimiting implementation, the catheter can be silicon, polyurethane, or Polytetrafluoroethylene (PTFE). The antimicrobial agent that coats the catheter can be silver, antimicrobial agents chlorhexidine, triclosan, or antibiotics like nitrofural, minocycline-rifampin in non-limiting examples. The antimicrobial agent in the coating can be eluting or non-eluting.

The catheter device provides a solution to the shortcomings in prior art through the provision of an antimicrobial exterior surface of catheter devices employed to communicate through the skin and to the body cavity. An antimicrobial surface area is positioned upon the catheter, such as along the entire length of the catheter or along one or more portions of the catheter. The one or more portions can be, for example, one or both distal ends of a catheter or a portion of the catheter that is located within the body cavity when the catheter is transcutaneously positioned in the body cavity. In this manner, during any communication of the catheter device through the skin, and into the patient, bacteria, viruses and other infectious occupants of the exterior and any underlying layers of skin, have direct contact with the antimicrobial coating.

In an implementation, the catheter uses bio-compatible surface coating in combination with the antimicrobial exterior surface coating. For example, by positioning a thin surface area of titanium on the exterior circumferential surface of polyurethane and other catheters in combination with an antimicrobial coating, the device and method herein renders conventional catheters into catheters with increased bio-compatibility as well as prevents the introduction of surface bacteria into the insertion site.

Any of a variety of antimicrobial materials may be employed. In addition to materials recited above, other example materials include one or a combination of antimicrobial materials from a group including nitrofurazone-coated silicone or silver or silver ions or silver nano-particles in a coating, or copper or copper bearing materials in a coating, chlorhexidine incorporated hydroxylapatite coatings, chlorhexidine-containing polylactide coatings on an anodized surface, and polymer and calcium phosphate coatings with chlorhexidine, and aluminum and aluminum ions. However, in other implementations the catheter may be impregnated or otherwise formed with antimicrobial materials and properties.

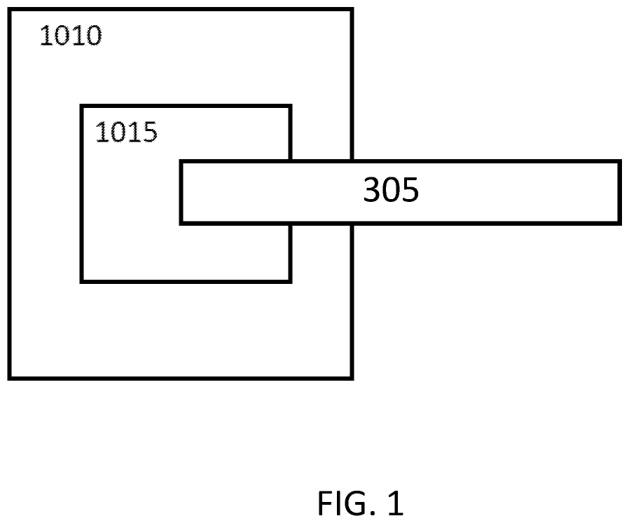

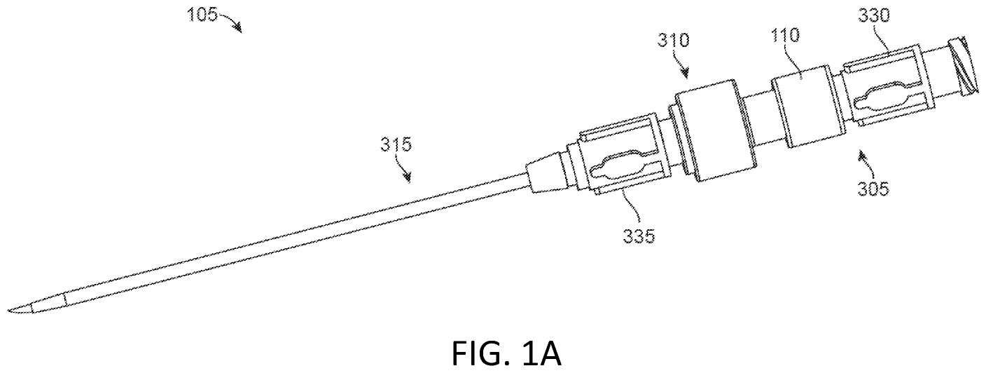

FIG. 1 shows a schematic representation of a catheter 305 inserted into a patient 101 such that the catheter 305 communicates with a body cavity 1015 of the patient 1010, wherein the body cavity 1015 is filled at least partially with fluid. The catheter 305 may be part of a catheter body access system 105 configured to allow fluid access to a body cavity (such as a pleural space in the body of a human or animal patient) from an extracorporeal location. The body cavity 1015 can be any fluid-filled cavity within the patient. In an embodiment, the body cavity 1015 is a pleural space of a patient. In another embodiment, the body cavity 1015 is a pre-existing cavity of a patient such that the cavity was not artificially formed or created by an intervention.

The catheter 305 is an elongated body having at least one internal lumen. The catheter 305 can be a cylindrical body with a single internal lumen that extends through the entire length of the catheter 305. In user, the distal end the catheter 305 is transcutaneously inserted into a patient's body and moved through the body so that the distal end is positioned within the body cavity 1015. Thus, the internal lumen of the catheter 305 fluidly communicates with the body cavity 1015. In this manner, the catheter 305 provides extracorporeal access to the body cavity 1015. Fluid of the body cavity 1015 can flow into the internal lumen of the catheter 305 and out of the catheter via an opening in the proximal end.

The catheter 305 can be formed of conventional materials such as polyurethane, silicone, or polytetrafluoroethylene (“PTFE”). However, it can also be formed of any material suitable for use in combination with the disclosed coating of an antimicrobial material for the purposes set forth in this disclosure. The catheter 305 can alternatively be formed all, or partially, itself of an antimicrobial impregnated material in a solid solution, or any other means to impart antimicrobial properties therein to communicate with the circumferential surface.

The antimicrobial coating may be applied by any conventional means known in the art such as vacuum chamber coating, plasma coating, employment of an antimicrobial material in an impregnated polymer or other carrier used as coatings, or impregnating the proximal and distal ends of the catheter itself, or other means which would occur to those skilled in the art. The antimicrobial materials could also include one or a combination of antimicrobial materials from a group including nitrofurazone-coated silicone or silver or silver ions or silver nano-particles, copper or copper bearing materials in a coating, or impregnated into the material forming the catheter, or shrunk wrapped. Other antimicrobial materials may be also placed adjacent in combination with the titanium such as one or a combination of, chlorhexidine incorporated hydroxylapatite coatings, chlorhexidine-containing polylactide coatings on an anodized surface, and polymer and calcium phosphate coatings with chlorhexidine, in a coating or mixed in a polymer coating.

In an embodiment, the antimicrobial feature is located at just the proximal portion or distal portion of the catheter 305. In the case of it being located at just a proximal portion, the antimicrobial portion protrudes from the insertion site communicating through the patient's skin, once the insertion procedure is complete. The placement of the coating on the proximal portion which communicates through the skin surface inhibits pathogen travel along the catheter surface and below the skin surface of the patient which is the first barrier to such intrusions.

The coating can be located on an exterior surface of the catheter, on an interior surface of the catheter, or on both the interior surface an exterior surface. In addition, the coating can be located on just a portion of the length of the catheter (such as along intermittent lengths of the catheter). In addition, the coating can be located and extend around an entire circumference of the catheter or along portions thereof either on the interior surface, the exterior surface, or both. The interior surface of the catheter can be a portion of the catheter that defines an internal lumen.

In another embodiment, the entire length of an exterior surface of the catheter 305 may additionally employ antimicrobial materials thereon singularly or in combination, or may have a titanium surface alone since it provides a means for encouraging lubricity. Alternatively, the titanium surface area on the entire circumferential surface may be employed in combination with one or a combination of the other noted antimicrobials herein.

In a non-limiting embodiment, the catheter 305 is configured pursuant to the body access system 105 described in FIGS. 1A-13, wherein the system includes a valved connector device that can be attached with and/or incorporated into a body fluid access device, such as a fluid drainage sheath or catheter. The valved connector includes a valve that automatically opens when a secondary device, such as a catheter or tubing, is properly attached to the valved connector. When opened, the valved connector permits fluid flow therethrough so that fluid can be drained from a location, such as a fluid cavity of a patient. The valved connector is closed in a default state and automatically transitions to the closed state when the secondary device is decoupled from the valved connector. In this manner, hemostasis through the body fluid access device in a default state.

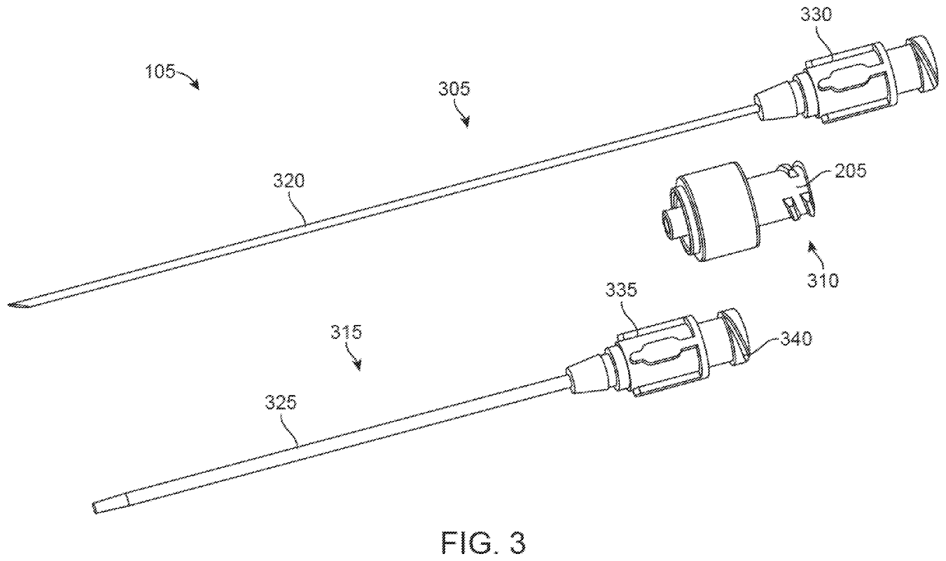

FIGS. 1A, 2, and 3 show a catheter body access system 105 configured to allow fluid access to a body cavity (such as a pleural space in the body of a human or animal patient) from an extracorporeal location. The system can also be used in a colorectal environment. The access system 105 includes a valve assembly or valved connector 310 that provides a regulated, fluid flow interface for a lumen of a body access sheath 315 (FIG. 3) or any device that is configured to access fluid within a body. The body access sheath 315 is an elongated device with an internal lumen that can be placed in extracorporeal communication with a fluid-filled body cavity or other location of a patient. The internal lumen of the body access sheath 315 provides a passageway for fluid to be drained out of the body cavity. The valved connector 310, when coupled to the body access sheath 315, regulates flow of fluid into and out of the lumen of the body access sheath 315. A secondary access device, such as a catheter 305 (FIG. 3), can be coupled to the body access sheath 315, as described below.

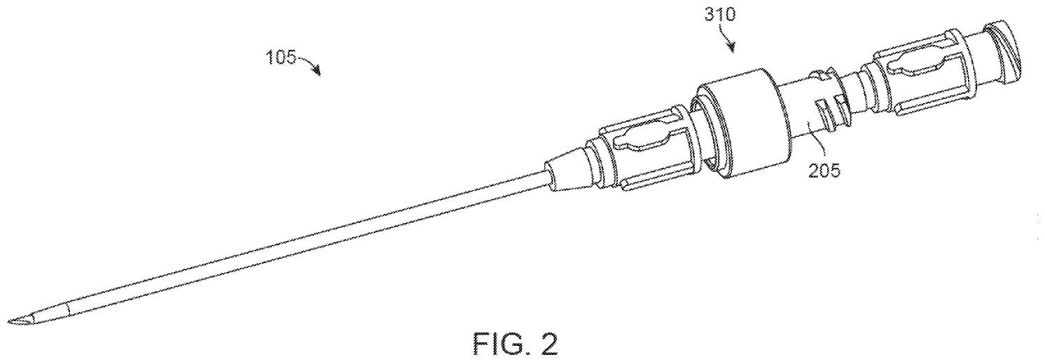

The valved connector 310, catheter 305, and sheath 315 are sized and shaped to be co-axially aligned and coupled to one another along a common long axis. FIG. 1A shows the valved connector 310, catheter 305, and sheath 315 in a coupled or assembled state so that the devices collectively form a unitary body. The devices include coupling mechanisms that permit a user to securely couple the devices to one another when co-axially aligned. For example, as shown in FIG. 1A, the catheter 305 includes a coupler 110, such as a Luer-type connector, that removably connects and secures to a portion of the valved connector 310 in order to secure the catheter 305 to the valved connector 310 such as via a threaded interface. FIG. 2 shows the system 105 with the coupler 110 removed from the catheter 305 in order to show a threaded interface 205 of the valved connector 310, wherein the threaded interface 205 attaches to the coupler 110 of the catheter 305.

FIG. 3 shows the access system 105 in an exploded state with the valved connector 310, catheter 305, and sheath 315 separated from one another. The catheter 305 includes an elongated body, such as a needle 320, that has a hub 330 on a proximal end. As mentioned, the hub 330 has a coupler 110 (shown in FIG. 1A) that can removably couple to a complementary threaded interface 205 of the valved connector 310. In the illustrated, example embodiment, the coupler 110 couples to the threaded interface 205 in a male-female relationship. The coupler 110 is rotatably attached to the hub 330 so that can rotate and threadedly attach to the threaded interface 205 of the valved connector 310.

With reference still to FIG. 3, the needle 320 of the catheter 305 has an internal lumen that runs the length of the needle 320 and that communicates with the hub 330. Fluid can flow into and out of the internal lumen of the needle 320 via a distal opening in the needle such as at a distal-most tip of the needle 320. An internal lumen of the hub 330 communicates with the internal lumen of the needle 320 and also communicates with a distal opening of the hub 330. In this manner, fluid can flow into the internal lumen of the needle and into the hub 330 and eventually out of the distal opening of the hub 330. The system 105 can further include a plug that inserts into the distal opening of the hub 330 to seal the opening closed.

As shown in FIG. 3, the sheath 315 includes an elongated cannula 325 with a cannula hub 335 on its proximal end. The cannula hub 335 has a threaded interface 340 that couples to a distal portion of the valved connector 310 such as in a threaded, male-female relationship. The cannula 325 of the sheath 315 has an internal lumen that runs the length of the cannula 325 and that communicates with the cannula hub 335. Fluid can flow into and out of the internal lumen of the cannula 325 via a distal opening in the cannula 325 such as at a distal-most tip of the cannula 325. An internal lumen of the hub 335 communicates with the internal lumen of the cannula 325 and also communicates with a distal opening of the hub 335. When the valved connector 310 is attached to the sheath 315, the valved connector 310 controls fluid flow through the internal lumen of the sheath 315, as described below.

The internal lumen of the cannula 315 is sized and shaped to co-axially receive the needle 320 of the catheter 305. In this manner, the sheath 315 and catheter 305 can be co-axially aligned when the needle 320 is inserted into the cannula 325. The needle 320 can have a length that is longer than the length of the cannula 325 so that a distal end of the needle 320 pokes out of the distal end of the cannula in the assembled device, such as shown in FIG. 1A.

As shown in FIG. 1A, the valved connector 310 can be positioned between the hub 335 of the sheath 315 and the hub 330 of the catheter 305. The valved connector lockingly secures to the hub 335 of the sheath 315 and the hub 330 of the catheter 305. When aligned and secured as such, the needle 320 of the catheter is positioned co-axially within the cannula 325 of the sheath with the valved connector 310 acting as a valved interface between the sheath 315 and the catheter 305.

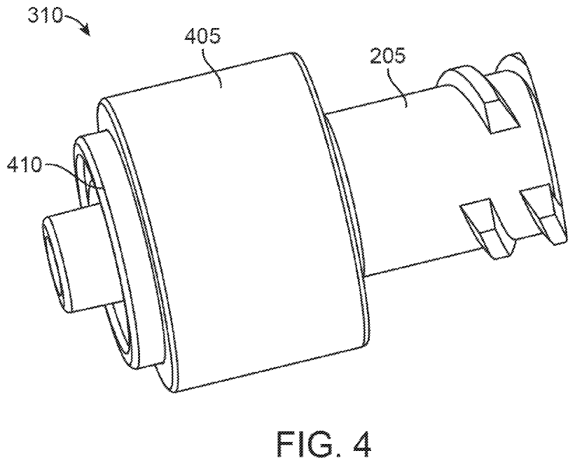

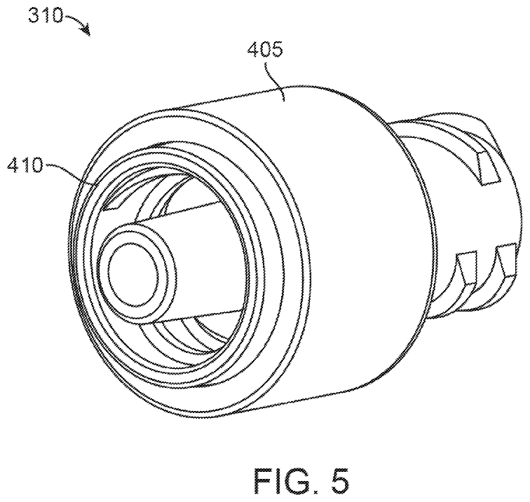

FIG. 4 shows a perspective view of the valved connector 310. FIG. 5 shows another perspective view of the valved connector 310. The valved connector 310 includes an outer housing 405 having a main body from which a neck extends. In the illustrated embodiment, the neck is circular in cross-section and has a smaller diameter than the diameter of a distal portion of the main body 405. The neck forms the threaded interface 205, which securely and sealingly couples to the coupler 110 (FIG. 1A) of the catheter 305 in a male-female relationship such as by rotating relative to one another. A distal interface 410 of the valved connector 310 is configured to couple to the threaded interface 340 (FIG. 3) of the cannula hub 335, as described further below. In the illustrated embodiment, the outer housing is substantially cylindrical although the shape may vary.

As shown in the proximal view of FIG. 5, the distal interface 410 of the valved connector has a threaded region (such as a threaded female surface), which is configured to couple in a rotatable, threaded manner to the threaded interface 340 (FIG. 3) of the cannula hub 335. That is, the threaded interface 340 inserts into the distal interface 410 of the valved connector and the two securely and sealingly couple to one another by a threaded engagement. When coupled as such, the internal lumen of the cannula 315 fluid communicates with an internal lumen of valved connector 310 such that fluid must flow through the valved connector in order to flow into or out of the internal lumen of the cannula 315 via the cannula hub 340.

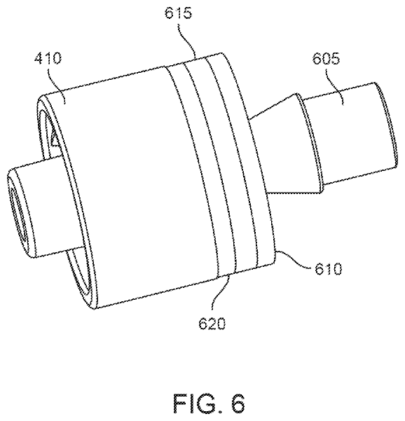

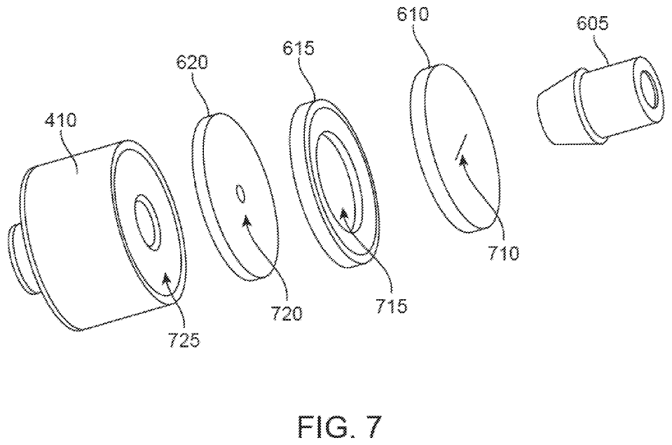

The outer housing 405 of the valved connector 310 defines an internal lumen that contains several components of the valved connector 310, wherein the components control fluid flow through the valved connector 310. FIG. 6 shows the internal components as they are arranged within an internal chamber of the outer housing 405 of the valved connector 310. The outer housing of the valved connector 310 is not shown in FIG. 6 for clarity of illustration. FIG. 7 shows the internal components in an exploded state.

With reference to FIGS. 6 and 7, the internal components include an elongated piston 605 having an internal lumen, which co-axially aligns with the long axis of the assembled system. The piston 605 has a substantially cylindrical shape with a sloped, distal end that reduces in transverse dimension (relative to the long axis) moving in a distal direction. That is, a diameter of the piston gradually reduces or tapers moving in a distal direction. In the assembled valved connector 310, the piston 605 is slidably positioned within the neck of the outer housing 405.

The internal components further include a round first seal disk 610 made of a malleable material that can seal with the piston 605 when in contact therewith. The first seal disk 610 has a slit 710 that extends through the seal disk 610. The seal disk is made of a material such that the slit seals shut in a default state and can also be deformed and/or forced open such as when contacted with sufficient force by the distal end of the piston. The slit 710 is sized to receive therethrough the needle 320 of the catheter 305.

The internal components further include a separator disk 615 positioned in a juxtaposed, contacting relationship with the first seal disk 610 and a second seal disk 620. The separator disk 615 is interposed between the first seal disk 610 and second seal disk 620 in the assembled state, as shown in FIG. 6. The separator disk 615 has a central aperture 715 with a chamfered, annular surface that surrounds the aperture 715 on both sides of the separator disk. The chamfered surface of the separator disk 615 is such that a space is formed in the region of the chamfered surface between both the second seal disk 620 and the separator disk 615, and also the first seal disk 610 and the separator disk 615. The chamfered surface can exist on one or both sides of the separator disk 615.

The second seal disk 620 also has a central aperture 720 that is sized and shaped to snugly receive therethrough the needle 320 of the catheter 305. The second seal disk 620 is also made of a resilient, flexible material.

With reference still to FIGS. 6 and 7, the distal interface 410 of the valved connector 310 has a front face 725 that is juxtaposed with the second seal disk 620 (in the assembled state) such that a central opening in the distal interface 410 co-axially aligns with the central apertures 720 and 715 and also with the slit 710. The distal interface 410, second seal disk 620, separator disk 615, and first seal disk 610 all have round outer shapes that fit snug within the main body 405 (FIGS. 4 and 5) in the assembled state. The piston 605 is slidably positioned within the neck of the main body 405 in the assembled state.

Although FIGS. 1A-3 show the access system 105 including a secondary access device comprising a catheter 305, it should be appreciated that variety of types and configurations of secondary access devices can be utilized in connection with valved connector 310. For example, the valved connector 310 can be attached to an access device that does not having a threaded connector. The valved connector 310 can also be secured to a catheter, tubing, or other apparatus or can be secured to an apparatus that is not directly in fluid communication with the volume of fluid in a body, but that is instead connected to a secondary apparatus. The valved connector can also be used to control the flow of fluid from one location to another location. For example, the valved connector can be used with an infusate bag or medical tubing that is not in communication with a patient's body. In another embodiment, the valved connector 310 can be an integral part of the sheath 315 such that the valved connector is not removable from the sheath 315. The valved connector 310 can also be attached upstream or downstream of the valved connector via tubing.

In use, the valved connector 310 is coupled to the sheath 315 such as by attaching the distal interface 410 of the valved connector 310 to the threaded interface 340 at the proximal end of the sheath 315. As mentioned, the distal interface 410 can be rotatably attached in a threaded manner to the threaded interface 340 to securely attach the valved connector 310 to the sheath 315. As mentioned, when attached as such, the internal lumen of the cannula 325 co-axially aligns with the internal lumen of the valved connector 310. In this manner, the valved connector 310 controls fluid flow (such as by inhibiting, permitting, or blocking flow) through the sheath 315 and valved connector based upon the state of the internal components of the valved connector. In an alternate embodiment, the valve connector 310 is integrally formed as part of the sheath 315.

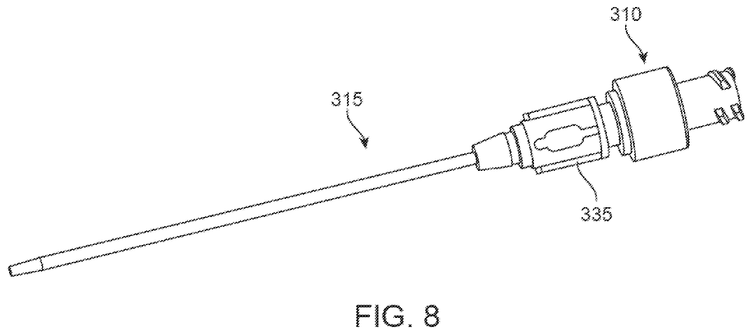

FIG. 8 shows the sheath 315 and the valved connector 310 attached to one another after the valved connector 310 has been coupled to the hub 335 of the sheath 315. As mentioned, the valved connector 310 attaches to the proximal end of the sheath 315 at the cannula hub 335. Once attached as such, the collective assembly of the sheath 315 and the valved connector 310 can be inserted by a user into fluid communication with a body cavity of a patient.

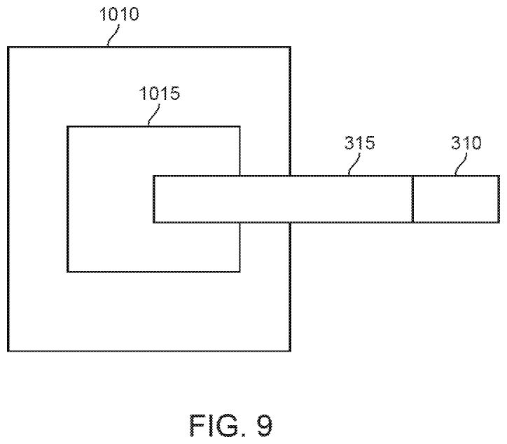

FIG. 9 shows a schematic representation of the collective assembly of the sheath 315 and the valved connector 310 inserted into a patient 101 such that the sheath 315 communicates with a body cavity 1015 of the patient 1010, wherein the body cavity 1015 is filled at least partially with fluid. The body cavity 1015 can be any fluid-filled cavity within the patient. In an embodiment, the body cavity 1015 is a pleural space of a patient. The distal end of the cannula of the sheath 315 is positioned within the body cavity 1015 such that the internal lumen of the sheath 315 fluidly communicates with the body cavity 1015. In this manner, the sheath 315 provides extracorporeal access to the body cavity 1015. Fluid of the body cavity 1015 can flow into the internal lumen of the sheath 315 wherein the valved connector 310 controls or regulates fluid flow out of the sheath 315. As mentioned, in a default state, the valved connector 310 is closed such that it blocks fluid from flowing out of the sheath 315. A lumen of the valved connector 310 is collectively formed by the central apertures 720 and 715 (FIG. 7) and the slit 710. This internal lumen of the valved connector 310 is initially closed as a result of bulk resilience of the material of the first disk 610 maintaining the slit 710 in a sealed, close state.

The valved connector 310 can be opened for fluid flow therethrough by coupling a secondary device, such as the catheter 305, to the valved connector 310. As the secondary device, such as the catheter 305, is inserted into the valved connector 310, a portion of the secondary device directly or indirectly deforms the slit 710 to thereby open the slit 710 and allow fluid flow therethrough. The process of coupling secondary device to the valved connector 310 and sheath 315 is now described in the example context of coupling the catheter 305 to the valved connector 310 and sheath 315.

The catheter 305 is coupled to the valved connector 310 by inserting the needle 320 of the catheter 305 into the internal lumen of the valved connector 310 so that the sheath 315, valved connector 310, and catheter 305 collectively form a single assembly, as shown in FIG. 1A. As mentioned, the various coupling components can be secured to one another to thereby secure the devices in an assembled state. The catheter 305 can be advance distally into and relative to the valved connector 310 and the sheath 315 such that the needle 320 advances into the internal lumen of the valved connector 310 and the internal lumen of the sheath 315. As the catheter 305 is coupled or advanced into to the valve connector 310, the catheter automatically transitions the valved connector 310 to the open state so that fluid can flow therethrough.

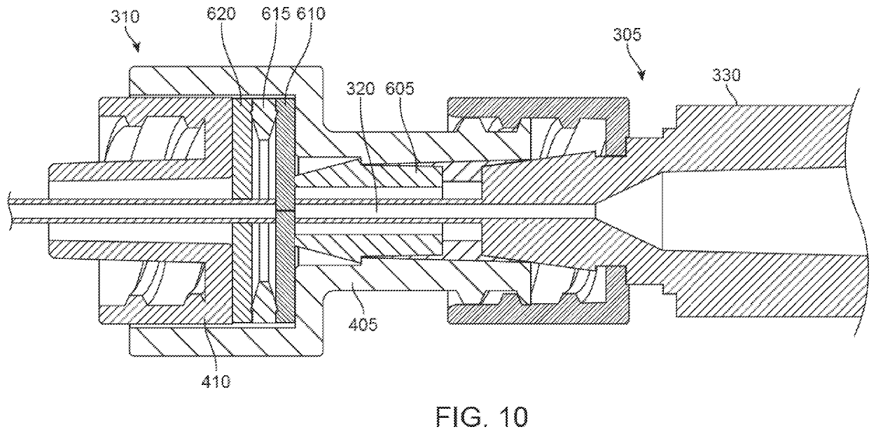

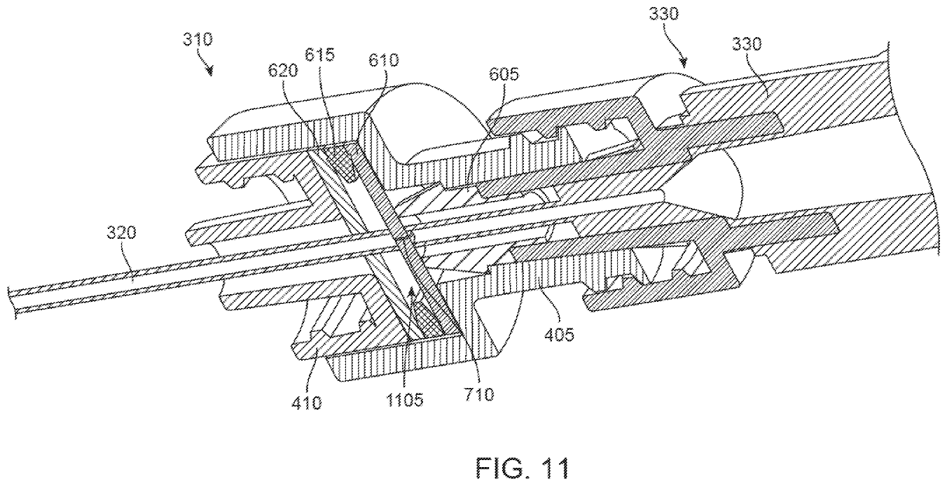

This is described in more detail with reference to FIGS. 10 and 11, which show cross-sectional view of the catheter access system 105 in the region of the valve connector 310 with the needle 320 of the catheter 305 inserted through the valved connector 310. The cannula 315 is not shown in FIGS. 10 and 11 for clarity of illustration. As the catheter 305 advances distally, a portion of the catheter 305 (such as a distal portion of the hub 330) abuts and pushes the piston 605 in a distal direction toward the first seal disk 610.

The distal end of the piston 605 moves distally (through the neck of the main body 405) so that it contacts the first seal disk 610. The distal end of the piston 605 exerts a force om the first seal disk 610 so as to deform the first seal disk 610 in a manner that opens the slit 710 (FIG. 11). As mentioned, the separator disk 615 is shaped so that a space 1105 (FIG. 11) is formed between the first seal disk 610 and the separator disk 615. This space provides for a region of movement and deformation of the first seal disk 610 as the piston 605 pushes against the first seal disk 506. The space 1105 is sufficiently large such that the first seal disk 610 can sufficiently deform within the space 1105 so that the slit 710 opens and provides a passageway for fluid to flow therethrough.

This creates a fluid passageway within the valved connector 310 through the slit 710 in a region that surrounds the needle 320 with the fluid passageway collectively formed by formed by the central apertures 720 and 715 (FIG. 7) and the slit 710 within the valved connector 310. Fluid can pass through this fluid passageway, which automatically opens upon coupling of the catheter 305 (or other secondary device) to the valved connector 310. Upon removal or de-coupling of the catheter 305 from the valved connector 310, the piston 605 automatically disengages from contact with the first seal disk 605. As mentioned, the first seal disk is made of a material such that the slit will automatically return to a closed state when the first seal disk is not deformed. In this manner, the first seal disk 610 and the valved connector 310 automatically close to prevent fluid flow therethrough upon removal of the catheter 305.

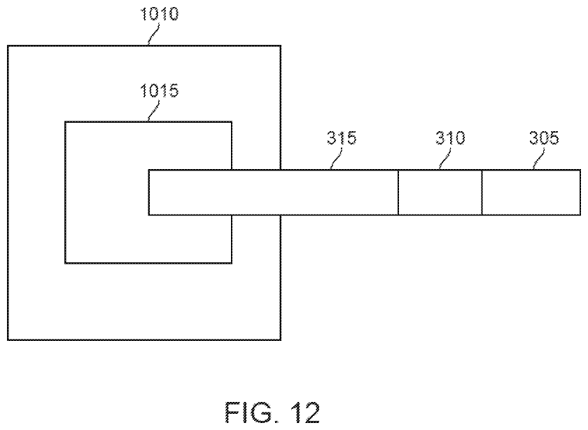

FIG. 12 shows a schematic representation of the collective assembly of the sheath 315, the valved connector 310, and the catheter 305 (or other such secondary access device) inserted into a body cavity 1015 of the patient 1010. As mentioned, the sheath 315 communicates with the body cavity 1015 such that the internal lumen of the sheath 315 can be used to drain fluid out of the body cavity 105. With the catheter 305 coupled to the valve connector 310, the valved connector 310 is automatically opened to permit fluid flow out of the sheath 315. As mentioned, other secondary devices aside from the catheter 305 can be coupled to the valved connector 310. The catheter 305 is just a non-limiting example.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. An antimicrobial catheter comprising:
an elongated body adapted to be inserted into a body cavity of a patient that is not vascular or the urethra, the elongated body having at least one interior lumen defined by a sidewall forming the elongated body, the elongated body having an exterior circumferential surface extending between a first end of the elongated body and a second end of the elongated body; and
an antimicrobial surface positioned along the circumferential surface, wherein the antimicrobial surface is configured to inhibit pathogens.

2. The antimicrobial catheter of claim 1, wherein the antimicrobial surface area extends along an entire length of the elongated body.

3. The antimicrobial catheter of claim 1, wherein the antimicrobial surface area extends along only a portion of a length of the elongated body.

4. The antimicrobial catheter of claim 1, wherein the antimicrobial surface comprises silicon, polyurethane or Polytetrafluoroethylene (PTFE).

5. The antimicrobial catheter of claim 1, wherein the antimicrobial surface comprises silver, chlorhexidine, or triclosan.

6. The antimicrobial catheter of claim 1, wherein the antimicrobial surface comprises nitrofural, minocycline-rifampin in non-limiting examples.

7. A method of draining fluid from a body cavity, comprising:
inserting a catheter into the body cavity, wherein the body cavity is a pre-existing cavity that is present prior to insertion of the catheter, and wherein the catheter comprises:
an elongated body adapted to be inserted into a body cavity of a patient, the elongated body having at least one interior lumen defined by a sidewall forming the elongated body, the elongated body having an exterior circumferential surface extending between a first end of the elongated body and a second end of the elongated body; and
an antimicrobial surface positioned along the circumferential surface, wherein the antimicrobial surface is configured to inhibit inhibiting pathogens;
using the catheter to drain fluid out of the body cavity.
